(19) United States
(12) Patent Application Publication
(10) Pub. No.: US 2014/0147411 A1
(43) Pub. Date: May 29, 2014

(54) METHODS OF TREATING CANCER AND TESTING MUTATION ZYGOSITY RELATED THERETO

(71) Applicants: U.S. GOVERNMENT represented by the UNITED STATES DEPARTMENT OF VETERANS AFFAIRS, Washington, DC (US); EMORY UNIVERSITY, Atlanta, GA (US)

(72) Inventors: Brian Paul Pollack, Decatur, GA (US); Bishu Sapkota, Atlanta, GA (US); Charles E. Hill, Atlanta, GA (US)

(73) Assignees: U.S. GOVERNMENT represented by the UNITED STATES DEPARTMENT OF VETERANS AFFAIRS, Washington, DC (US); EMORY UNIVERSITY, Atlanta, GA (US)

(21) Appl. No.: 14/077,348

(22) Filed: Nov. 12, 2013

Related U.S. Application Data
(60) Provisional application No. 61/726,030, filed on Nov. 14, 2012.

(51) Int. Cl. A61K 39/395; A61K 38/21; A61K 31/506; A61K 35/12; A61K 31/44; A61K 31/495; A61K 39/00; A61K 38/20; A61K 31/437

(52) U.S. Cl. CPC A61K 39/39558; A61K 38/2013; A61K 38/217; A61K 38/215; A61K 38/212; A61K 31/506; A61K 31/437; A61K 31/44; A61K 31/495; A61K 39/3955; A61K 39/0011; A61K 35/35
USPC 424/85.2; 424/85.5; 424/85.6; 424/85.7; 424/130.1; 514/275; 514/300; 514/350; 514/393; 424/277.1; 424/93.7

(57) ABSTRACT

In certain embodiments, the disclosure relates to methods of treating cancer comprising administering an effective amount a tyrosine kinase inhibitor in combination with an immunotherapy to a subject in need thereof. In certain embodiments, the disclosure relates to methods of treating cancer comprising: i) analyzing both chromosomes in a cell from a subject for the a V600E mutation of BRAF; and ii) determining if both of the chromosomes contain the V600E mutation, then treating the subject comprising the step of administering an effective amount a tyrosine kinase inhibitor in combination with an immunotherapy to the subject.

### CROSS REFERENCE TO RELATED APPLICATIONS

This application claims priority to U.S. Provisional Application No. 61/726,030 filed Nov. 14, 2012, hereby incorporated by reference in its entirety.

### BACKGROUND

A BRAF V600E mutation is associated with malignant melanomas. See Davies et al., Nature, 2002, 417, 949-954. Chapman et al., report an improved survival with vemurafenib in melanoma in patients with a BRAF V600E mutation. N Engl J Med 2011; 364:2507-164. While many patients whose tumors harbor the BRAFV600E mutation respond initially to kinase inhibitors such as vemurafenib, the development of resistance is common, and long-term complete responses (CR) are less than optimal. As such, additional approaches to treat those with advanced melanoma are still needed.

INTRON® A is recombinant Interferon alfa-2b. It is adjuvant to surgical treatment in patients with melanoma at high risk for systemic recurrence. See FDA Package Insert.

A clinical trial of the combination of drugs vemurafenib and aldesleukin (IL-2) is contemplated. See http://clinicaltrials.gov/show/NCT01754376.

A clinical trial for treating melanoma by lymphodepletion plus adoptive cell transfer and IL-2 in combination with ipilimumab is contemplated.

See http://clinicaltrials.gov/show/NCT01701674.

Rubinstein et al. report incidence of the V600K mutation among melanoma patients with BRAF mutations, and potential therapeutic response to the specific BRAF inhibitor PLX4032. J Trans Med, 2010, 8:67. See also Sigalotti et al., Br J Cancer. 2011; 105:327-8; Sapkota et al., Oncolmmunology, 2013, 2:1, e22890; Importa et al., Oncolmmunology, 2013, 2:8, e25594; WO2012075327; WO2007002811; WO2013044169; WO2012109329; WO2011093606; CA 2761253; and US 20120244151.

The Cobas® 4800 BRAF V600 Mutation Test is a real-time PCR based method to detect BRAF V600E mutations in DNA isolated from formalin-fixed, paraffin-embedded human melanoma tissue. Other techniques include bidirectional direct Sanger sequencing (“Sanger”) and the Applied Biosystems BRAF Mutation Analysis Reagents kit (“FA test”) for the detection of BRAF V600 mutations in formalin fixed paraffin embedded (FFPE) specimens. See Lopez-Rios et al., PLOS ONE available at www.plosone.org, 2013, 8(1):e53733, Machnicki et al., Acta Biochim Pol., 2013, 60(1):57-64.

Liu et al. report stat3-targeted therapies overcome the acquired resistance to vemurafenib in melanomas. J Inv Derma, 2013, 133, 2041-2049.

References cited herein are not an admission of prior art.

### SUMMARY

In certain embodiments, the disclosure relates to methods of treating cancer comprising administering an effective amount a tyrosine kinase inhibitor in combination with an immunotherapy to a subject in need thereof. In certain embodiments, the disclosure relates to methods of treating cancer comprising: i) analyzing both chromosomes in a cell from a subject for the a V600E mutation of BRAF; and ii) determining if both of the chromosomes contain the V600E mutation, then treating the subject comprising the step of administering an effective amount a tyrosine kinase inhibitor in combination with an immunotherapy to the subject.

In certain embodiments, the cancer is melanoma.

In certain embodiments, the tyrosine kinase inhibitor is vemurafenib, PLX4720, sorafenib, temozolomide, or dabrafenib.

In certain embodiments, the immunotherapy is administration of an interferon, administration of an interleukin, administration of an anti-PD1 or PD-L1 antibody, administration of an anti-CTLA-4 antibody, a cancer vaccine, adoptive cell transfer, and combinations thereof.

In certain embodiments, the interferon is human recombinant IFN-α, IFN-α2b or IFN-γ or the interleukin is human recombinant IL-2.

In certain embodiments, methods disclosed herein comprise the step of recording whether the subject is homozygous for the V600E mutation, heterozygous for the V600E mutation, heterozygous for the wild-type and V at 600, or homozygous for the wild-type V at 600.

In certain embodiments, the recording is on a computer readable medium.

In certain embodiments, methods disclosed herein comprise the step of reporting whether the subject is homozygous for the V600E mutation, heterozygous for the V600E mutation, heterozygous for the wild-type V at 600, or homozygous for the wild-type V at 600 to a medical professional, subject, or representative thereof.

### BRIEF DESCRIPTION OF THE FIGURES

FIG. 1 shows data indicating the BRAFV600E selective inhibitor PLX4720 enhances the induction of MHC class I molecules in A375 melanoma cells. A, PLX4720 decreases levels of phospho-ERK. A representative western blot is shown. A375 cells were pre-treated with vehicle (DMSO) or 10 μM of PLX4720 60 minutes prior to the addition of IFN-γ (100 U/ml). Whole cell lysates were prepared 24 hours later and levels of phospho-ERK (Thr 202/Tyr 204) and total ERK protein were analyzed by western blot. B, A representative flow cytometry histogram is shown. A375 cells were pre-treated with vehicle (DMSO, black line, unfilled) or 10 μM PLX4720 (black dotted line) for 60 minutes prior to the addition of IFN-γ (10 U/ml). Control cells were left untreated (gray filled). Cell surface MHC class I was analyzed using flow cytometry 72 hours later using an antibody that recognizes a shared epitope on HLA-A, B and C molecules. Cells stained with an isotype control antibody are shown (black filled). C, Averaged mean fluorescence intensity (MFI) from three independent flow cytometry experiments is shown (y-axis) and treatments are indicated along the x-axis. The error bars represent the SEM. (*, p<0.05; two-tailed paired Student's t test, as compared to cells treated with IFN-γ and pre-treated with DMSO).

FIG. 2 shows data indicating the enhancement of MHC induction by vemurafenib increases with escalating concentrations of IFN-γ and is associated with increased CIITA and NLRC5 expression. A, Vemurafenib decreases levels of phospho-ERK in A375 cells. A representative western blot is shown. A375 cells were treated with vehicle (DMSO) or vemurafenib (10 μM, VEM) alone or in combination with IFN-γ (2000 U/ml). Cell lysates were prepared 72 hours later and levels of total ERK and phospho-ERK (Thr 202/Tyr 204) protein analyzed by western blot. B, A375 (BRAFV600E homozygous) or SKMEL-2 (BRAF codon 600 wild-type) cells were pretreated with vehicle (DMSO, gray diamonds) or vemurafenib (10 μM, black squares) 60 minutes prior to the addition of IFN-γ at concentrations indicated along the x-axis. Cell surface MHC class I (HLA-A, B, C), B2M, and MHC class II (HLA-DR) levels were analyzed 72 hours later using flow cytometry. Values represent the average mean fluorescence intensity (MFI) for three independent experiments and error bars represent the SEM. (*, p<0.05, *** p<0.001, repeated measures ANOVA, as compared to DMSO-pre-treated cells exposed to the same concentration of IFN-γ) C, A representative western blot of CIITA protein levels from A375 cells is shown. Protein lysates were isolated from A375 cells 72 hours after treatment with vehicle (DMSO) or vemurafenib as the only treatment or 60 minutes prior to the addition of IFN-γ (2000 U/ml). GAPDH levels are shown as a loading control. D, Induction of HLA-A, HLA-DR, CIITA and NLRC5 mRNA in A375 cells. A375 cells were pre-treated with vehicle (DMSO) or vemurafenib (10 μM) 60 minutes prior to the addition of IFN-γ (2000 U/ml). Control cells were treated only with vehicle (DMSO). Steady state mRNA levels were measured using quantitative real-time RT-PCR 72 hours later and are expressed as fold over vehicle (DMSO) treated cells. Error bars represent SEM from at least 3 independent experiments. (*, p<0.05, **, p<0.01, 2-tailed paired Student's t test, as compared to cells treated with IFN-γ and pre-treated with DMSO)

FIG. 3 shows data indicating nanomolar concentrations of vemurafenib enhance the induction of MHC class I, β2-microglobulin and MHC class II molecules on A375 cells. A, Representative flow cytometry histograms are shown for cell surface expression of MHC class I (HLA-A,B,C; left panel), β2-microglobulin (B2M, middle panel) or MHC class II (HLA-DR, right panel) on A375 cells treated with vehicle alone (DMSO, gray filled), treated with vehicle 60 minutes prior to the addition of IFN-γ (2000 U/ml, black line), or vemurafenib (625 nM) 60 minutes prior to the addition of IFN-γ (2000 U/ml, black dotted line). Cells stained with an isotype control antibody are shown (black filled). B, The average MFI from 3 independent flow cytometry experiments is shown along the y-axis. A375 cells were treated with vehicle (DMSO) alone (1st bar) or 60 minutes prior to the addition of IFN-γ (2000 U/ml, 2nd bar). The 3rd through the 9th bars represent cells pre-treated with vemurafenib at the concentrations indicated along the x-axis 60 minutes prior to IFN-γ (2000 U/ml). Cells pre-treated with dacarbazine (DTIC, 20 μM), sorafenib (SOR, 10 μM), and temozolomide (TEMO, 10 μM) 60 minutes prior to IFN-γ (2000 U/ml) are shown in the 10th-12th bars respectively as indicated along the x-axis. Error bars represent the SEM. (*, p<0.05, ** p<0.01, ***, p<0.001, repeated measures ANOVA, as compared to DMSO-pre-treated cells exposed to the same concentration of IFN-γ) C, Forced over-expression of BRAFV600E in A375 cells decreases MHC class I expression. A375 cells were transiently transfected with a plasmid encoding BRAFV600E and green fluorescent protein (GFP) or empty vector encoding GFP alone. Flow cytometry was used to select transfected (GFP positive) and non-transfected (GFP negative) cells and MHC class I levels were measured on these two cell populations. Average values from three independent experiments are shown. MHC class I levels are expressed as the % of MHC class I on control cells (cells non-transfected using the empty vector plasmid). (***, p<0.001, repeated measures ANOVA, as compared to non-transfected cells) D, Representative flow cytometry histograms are shown for untreated A375 cells (gray filled), or those pre-treated with vehicle (DMSO, solid black line) or vemurafenib (500 nM, dotted black line) 60 minutes prior to IFN-α2b (909 U/ml). Cells were stained for MHC class I (left panel, HLA-A,B,C) or MHC class II (right panel, HLA-DR) 72 hours following the addition of IFN-α2b. Cells stained with an isotype control antibody are shown in black filled. E, The average MFI from five experiments is shown for A375 cells pre-treated with vehicle (gray diamonds) or vemurafenib (500 nM, black squares) for 60 minutes prior to the addition of IFN-α2b at the doses shown along the x-axis. Error bars represent the STDEV (***, p<0.001, repeated measures ANOVA, as compared to cells treated with the same concentration of IFN-α2b and vehicle)

FIG. 4 shows data indicating vemurafenib enhancement of MHC induction occurs in cell lines harboring only a BRAFV600E mutation and not those with heterozygous BRAFV600E mutations. A, Vemurafenib enhances the induction of MHC class I (HLA-A, B and C) and MHC class II molecules in cell lines homozygous for BRAFV600E. The cell lines indicated above the panels were pre-treated with either vehicle (DMSO, gray diamonds) or vemurafenib (0.5 μM, black squares) and then treated with IFN-γ at the concentrations indicated along the x-axis. Cell surface MHC class I (HLA-A, B and C, top panels) and MHC class II (HLA-DR, lower panels) levels were measured 72 hours later using flow cytometry. The y-axis represents average MFI for at least three independent experiments except those for MeWo and SKMEL-5 which are the average of two independent experiments. Error bars represent the SEM. (*, p<0.05, ** p<0.01, repeated measures ANOVA, as compared to DMSO-pre-treated cells exposed to the same concentration of IFN-γ) B, The impact of vemurafenib on fold MHC class I induction in melanoma cell harboring heterozygous and homozygous BRAFV600E mutations. Fold induction of cell surface MHC class I was calculated by dividing averaged MFI values for cells treated as indicated along the x-axis by averaged MFI values of cells treated with vehicle (DMSO) alone. Fold inductions for heterozygous cell lines (MALME-3M, SKMEL-3, and SKMEL-5) were averaged together (gray bars) as were the fold inductions for three homozygous (white bars) cell lines (A375, HT-144, and SKMEL-28). (**, p<0.01, ***, p<0.001, repeated measures ANOVA, as compared to identically treated BRAFV600E heterozygous cells; ††, p<0.01, †††, p<0.001, repeated measures ANOVA, as compared to BRAFV600E homozygous cells pre-treated with DMSO and exposed to the same concentration of IFN-γ) C, Vemurafenib increases CIITA steady state mRNA levels in SKMEL-28 cells. SKMEL-28 cells were pre-treated with vehicle (DMSO) or vemurafenib (0.5 μM) for 60 minutes prior to the addition of IFN-γ (20 U/ml). CIITA steady state mRNA levels were assessed at the time points indicated along the x-axis and are expressed as fold induction over cells treated only with vehicle (DMSO). (***, p<0.001, repeated measures ANOVA, as compared to DMSO-pre-treated cells exposed to the same concentration of IFN-γ) D, The dual PI3K/mTOR inhibitor does not enhance the induction of MHC molecules by IFN-γ. The DMSO-treated cells (gray diamonds) from part A are compared to those treated with BEZ235 (0.5 μM, black squares). MHC class I and class II levels were assessed as in part A.

FIG. 5 illustrates interactions between BRAFV600E, immune gene expression and anti-tumor immune responses. BRAFV600E has a repressive effect on MHC expression such that the induction of MHC molecules by IFN-γ or IFN-α2b can be enhanced in the presence of BRAFV600E-inhibitors. Increases in MHC expression are likely complemented by the increases in melanocyte differentiation antigens (MDA) that are induced by inhibitors of BRAFV600E. Enhanced MHC expression can increase the recognition of tumor cells by intra-tumoral T cells which are augmented in the setting of vemurafenib therapy. BRAFV600E can increase the expression of cytokines such as interleukin (IL)-1α/β that can promote the immunosuppressive effects of tumor associated fibroblasts. These effects can be blocked by BRAFV600E inhibitors.

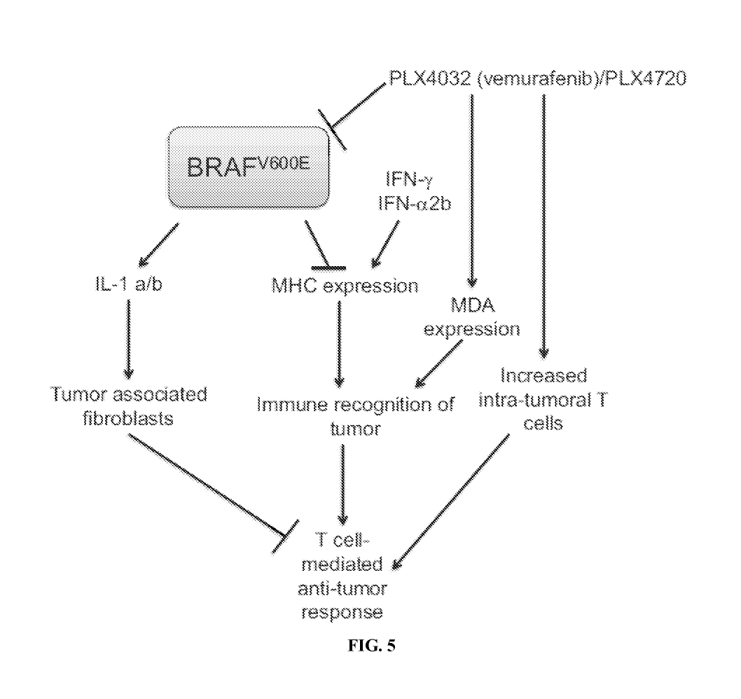

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

As used herein, the term “combination with” when used to describe administration with an additional treatment means that the agent may be administered prior to, together with, or after the additional treatment, or a combination thereof.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the condition or disease is eradicated. Rather, embodiments, of the present disclosure also contemplate treatment that merely reduces symptoms, and/or delays conditions or disease progression.

### Vemurafenib Enhances MHC Induction in BRAFV600E Homozygous Melanoma Cells

Ipilimumab has been approved by the Food and Drug Administration (FDA) for the treatment of those with metastatic melanoma. It is a human monoclonal antibody that activates the immune system by targeting CTLA-4. Other immune-based approaches are under investigation for the treatment of cancer. These methods attempt to enhance the development of anti-tumor CD8+ cytotoxic lymphocytes (CTLs) and CD4+ T lymphocytes to generate a therapeutic cell-mediated anti-tumor immune response. Immune cell-mediated responses are believed to involve the processing and presentation of antigenic peptides bound to MHC molecules which allow their recognition by CTLs and/or CD4+ T cells. Enhancing MHC expression on tumor cells is believed to be a means of improving tumor cell immune recognition. See Lampen & Hall T, Cur Opin Immunol, 2011; 23:293-8. Rosenberg et al. report adoptive cell transfer is a clinical path to effective cancer immunotherapy. Nat Rev Cancer 2008; 8:299-308.

IFNs are potent inducers of MHC expression. The influence BRAF V600E inhibitors on the induction of MHC molecules by interferons (IFNs) were explored. It has been discovered that vemurafenib can enhance the induction of MHCI and MHCII molecules by IFN-γ and IFN-α2b in A375 melanoma cells. Vemurafenib could enhance MHC induction by IFN-γ in melanoma cells harboring a homozygous BRAFV600E mutation but not in cell lines heterozygous for BRAFV600E or those wild-type at BRAF codon 600. These data suggest that inhibition of BRAFV600E can enhance MHC induction by IFNs in some cellular contexts and supports the notion that the impact of vemurafenib on immune gene expression can be influenced by the zygosity of the BRAFV600E mutation.

The data presented herein demonstrate that BRAFV600E has a repressive effect on MHC expression and that in some cellular contexts, inhibition of BRAFV600E activity can augment the induction of MHCI and MHCII molecules by IFN-γ, a cytokine that is likely to be present in the tumor microenvironment, and IFN-α2b. This finding is relevant for several reasons. Most notably, the expression of MHCI molecules in melanoma has been shown to correlated to the clinical response to immune-based therapies. See Carretero et al., Immunogenetics 2008; 60:439-47 and Carretero et al., Int J Cancer 2012; 131:387-95. These results indicate that BRAFV600E inhibition alone or combined with IFN-α2b is a promising pharmacologic approach to enhance the expression of MHCI molecules on melanoma cells. It supports the notion that combining IFN-α2b and BRAFV600E inhibition may offer an approach to augment tumor cell immune recognition by CTLs in the adjuvant setting. Data herein indicates that in addition to providing a growth advantage to tumor cells, the genetic amplification of BRAFV600E may also promote tumor cell immune escape by attenuating basal MHCI levels.

Because of its pivotal role in regulating cell proliferation, the prevailing paradigm regarding the BRAFV600E mutation in melanoma has centered on whether the mutation is there or not. If the BRAFV600E mutation is present, as measured via sequencing or mutation-specific PCR-based assays, patients are eligible to be placed on a BRAFV600E-selective inhibitor such as vemurafenib or dabrafenib. However, less attention has been given to the zygosity of the BRAFV600E mutation. This is primarily because there has been no clinical need to differentiate BRAFV600E heterozygous tumors from BRAFV600E homozygous tumors or understand how the zygosity of the mutation influences tumor biology.

At present, molecular testing for the use of inhibitors that are selective for the BRAFV600E mutation centers on the detection of BRAFV600E in DNA isolated from patient tumor samples. Typically, this is biopsy material that has been formalin-fixed and paraffin embedded. Indeed, a test for this purpose has been approved by the FDA. However, in these assays, the zygosity of the BRAFV600E mutation is not routinely assessed. Our data raise the possibility that the zygosity of BRAFV600E mutation may influence how melanoma cells respond to vemurafenib (or other targeted inhibitors of BRAFV600E) with regards to the expression of MHC molecules, immune system genes directly relevant to anti-tumor immune responses in melanoma. This is particularly important as combination therapies utilizing both targeted kinase inhibitors and immune-based approaches in patients with advanced melanoma. BRAFV600E zygosity is a relevant biomarker for therapies using BRAFV600E-specific kinase inhibitors alone or in combination with an immune-based therapeutic (such as IL-2 or ipilimumab).

BRAFV600E can influence basal MHCI expression and that inhibitors of BRAFV600E can potentiate the induction of MHC molecules by IFN-γ and IFN-α2b. This effect is believed to be mediated via a mechanism that is influenced by the zygosity of the BRAFV600E mutation.

### Analyzing Chromosomes for the a V600E Mutation

Analyzing chromosomes for the V600E mutation may be performed by any current method known to the skilled artisan. One method is to isolate a malignant melanocyte, optionally separating the chromosomes within the cell, and sequencing the target DNA region, e.g., using PCR, to determine the presence of the mutation or wild-type sequence.

Genomic DNA was isolated from the cell lines and BRAF codon 600 was amplified using PCR and sequenced. The wild-type BRAF codon 600 sequence is GTG whereas the BRAFV600E codon sequence is GAG. A431 cells are wild-type as are melanoma cell lines MeWo and SKMEL-2. Cell lines heterozygous for BRAFV600E (SKMEL-3, SKMEL-5, and MALME-3M) have a mixture of T and A at the second nucleotide position of codon 600 whereas cell lines harboring only BRAFV600E (A375, SKMEL-28, and HT-144) have only the GAG sequence at codon 600.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). Any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

### Combination Therapies

In certain embodiments, the disclosure relates to methods of treating cancer comprising: i) analyzing both chromosomes in a cell from a subject for the a V600E mutation of BRAF; and ii) determining if both of the chromosomes contain the V600E mutation, then treating the subject comprising the step of administering an effective amount a tyrosine kinase inhibitor in combination with an immunotherapy to the subject.

In certain embodiments, the tyrosine kinase inhibitor is vemurafenib, PLX4720, sorafenib, temozolomide, trametinib, or dabrafenib.

In certain embodiments, the immunotherapy is administration of an interferon, administration of an interleukin, administration of an anti-PD1 or PD-L1 antibody, administration of an anti-CTLA-4 antibody, a cancer vaccine, adoptive cell transfer, and combinations thereof.

In certain embodiments, the interferon is human recombinant IFN-α, IFN-α2b or IFN-γ or the interleukin is human recombinant IL-2 or IL-12 or fragments thereof.

In certain embodiments, the disclosure relates to methods of treating the subject comprising the step of administering an effective amount a tyrosine kinase inhibitor in combination with a cancer vaccine. In certain embodiments, the cancer vaccine comprises cancer antigens, MAGE-A3, MART-1, gp100, TRP-2, NY-ESO-1, costimulatory molecules, cytokines, and combinations thereof.

In certain embodiments, the cancer antigen or combinations of antigens are expressed on the surface of virus particle or virus-like particles. The virus particles may be the result of genetically engineered attenuated or weakened viruses that produce virus or virus-like particles containing the antigen.

In certain embodiments, the cancer vaccine is a recombinant virus that expresses cytokines or cancer antigens. Willomann et al., report expression of IFN-beta enhances oncolytic vesicular stomatitis virus for therapy of mesothelioma. Cancer Res. 2009 Oct. 1; 69(19):7713-20. In certain embodiments, the cancer vaccine in is a recombinant vesicular stomatitis virus that expresses IFN-beta. Wollmann et al. report vesicular stomatitis virus variants selectively infect and kill human melanomas but not normal melanocytes. J Virol., 2013, 87(12):6644-59. In certain embodiments, the cancer antigen containing virus like particles may also contain additional adjuvants, e.g., flagellin, GM-CSF, on the surface of the particle. Wang et al., report incorporation of membrane-anchored flagellin into influenza virus-like particles enhances the breadth of immune responses. J Virol., 2008, 82(23): 11813-11823.

In certain embodiments, the cancer antigen or combinations of antigens are expressed on the surface of cells. The cells may be the result of genetically engineering cells to express the antigen on the surface. In certain embodiments, the antigen is anchored on the surface of the cell through glycosyl phosphatidylinositol (GPI), e.g., as a result of mixing cells with an antigen conjugate with glycosyl phosphatidylinositol. The cancer antigen containing particles may also contain additional adjuvants, cytokines, or costimulatory molecules. See Bozeman et al., Vaccine, 2013, 31(20):2449.

In certain embodiments, the disclosure relates to methods of treating a subject comprising the step of administering an effective amount a tyrosine kinase inhibitor in combination with adoptive cell transfer. Adoptive cell transfer relates to the isolation, amplification, optionally modification, and reinfusion of cells of the immune system cells, e.g., T lymphocytes, NK cells. In certain embodiments, the disclosure relates to methods using adoptive cell transfer (ACT) with autologous tumor-infiltrating lymphocytes (TILs) or using tumor-infiltrating lymphocytes (TILs) or T lymphocytes obtained from bone marrow or peripheral blood and expanded ex vivo. In certain embodiments, the disclosure relates to using autologous TILs expanded ex vivo from tumor fragments or single cell enzymatic digests of melanoma metastases.

In certain embodiments, the disclosure relates to methods wherein the subject undergoes chemotherapy causing lymphodepletion, e.g., by administering chemotherapy agents such as cyclophamide and flubarabine.

In certain embodiments, the disclosure relates to methods using peripheral blood mononuclear cells (PBMCs) obtained by leukapheresis. In certain embodiments, the disclosure relates to methods infusing tumor-specific CD4+ and CD8+ T cells generated in vitro with repeated stimulation of irradiated autologous tumor cells. In certain embodiments, the disclosure relates to methods using PBMC stimulated with artificial antigen-presenting cells, costimulatory molecules, cytokines, and combinations thereof.

In certain embodiments, the disclosure relates to methods using T cells engineered to stably express transgenes by vector based transduction. Vector mediated gene transfer approaches may use vectors that are derived from viruses, e.g., gamma retroviruses, lentiviruses or foamy virus vectors, that have the ability to integrate into the cell genome and provide transgene expression. Transduction may be through the use of replicating cells for viral integration into genomic DNA or nondividing cells. Bauer et al., report a foamy virus vector. Nature Medicine, 2008, 14, 93-97 (2008).

In certain embodiments, the cell therapy comprises modifying autologous cells, e.g., dendritic cells, genetically modified to produce IL-2, IL-12, or interleukin-12p70 (IL-12p70). Carreno et al. report genetically modified dendritic cells producing IL-12p70 elicit Tcl-polarized immunity. J Clin Invest. 2013, 123(8):3383-94

In certain embodiments, the methods disclosed herein comprise using a tumor antigen expressing recombinant vesicular stomatitis virus in combination with an adoptive cell transfer therapy. Rommelfanger et al. report a systemic combination virotherapy for melanoma with tumor antigen-expressing vesicular stomatitis virus and adoptive T-cell transfer. Cancer Res., 2012, 15; 72(18):4753-64.

In certain embodiment, methods disclosed herein comprise viral gene transfer into autologous cells of a drug-resistant enzyme such as P140KMGMT for reinfusion and subsequent treatment with temozolomide. Dasgupta et al. report engineered drug-resistant immunocompetent cells enhance tumor cell killing during a chemotherapy challenge. Biochem Biophys Res Commun., 2010, 391(1):170-5.

In certain embodiments, the disclosure relates to methods of treating cancer comprising: i) analyzing both chromosomes in a cell from a subject for the a V600E mutation; and ii) determining if both of the chromosomes contain the V600E mutation, then treating the subject comprising the step of administering an effective amount a tyrosine kinase inhibitor and an immune therapy in combination with a chemotherapy agent, e.g., imatinib, nilotinib, orafenib, bevacizumab, pazopanib, everolimus, and combinations thereof.

### Examples

### PLX4720 Enhances the Induction of MHCI by IFN-γ in A375 Cells

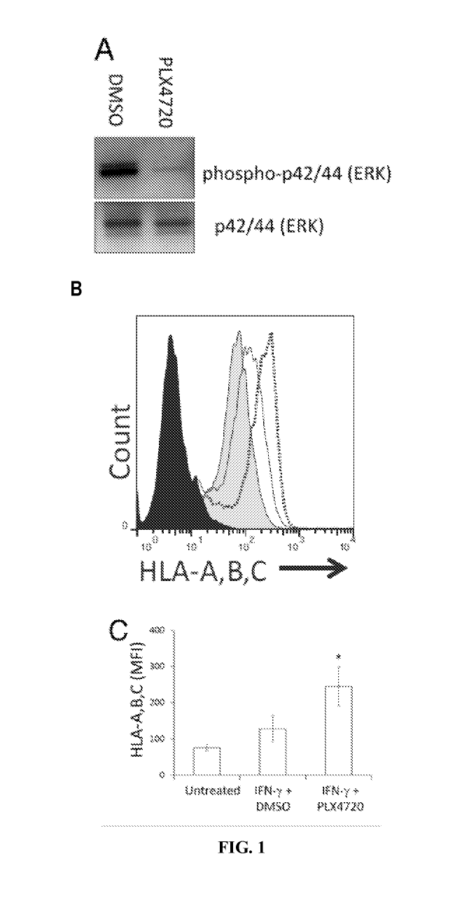

Experiments were performed to determine whether inhibitors of BRAFV600E could potentiate the effects of IFN-γ on MHC expression. A375 cells were selected as a model tumor cell line since A375 cells are known to respond to IFN-γ and harbor the BRAFV600E mutation. To confirm that PLX4720 inhibits BRAFV600E signaling, A375 cells were treated with either vehicle (DMSO) or 10 μM PLX4720 and evaluated levels of ERK phosphorylation (at residues threonine 202 and tyrosine 204) as a read out for activated MAPK signaling. As shown in FIG. 1A, PLX4720 reduced levels of ERK phosphorylation.

Experiments were performed to examine whether PLX4720 could influence the induction of MHCI molecules by IFN-γ in A375 cells. Treatment of A375 cells with IFN-γ lead to an induction of MHCI cell surface expression as measured by flow cytometry (FIGS. 1B and C). Pre-treatment of A375 cells with PLX4720 enhanced the induction of MHCI molecules by IFN-γ suggesting that BRAFV600E inhibition can influence MHCI induction by IFN-γ in some melanoma contexts (FIGS. 1B and 1C).

### Vemurafenib Enhances the Induction of MHCI, β2M and MHCII Molecules by IFN-γ in A375 Cells

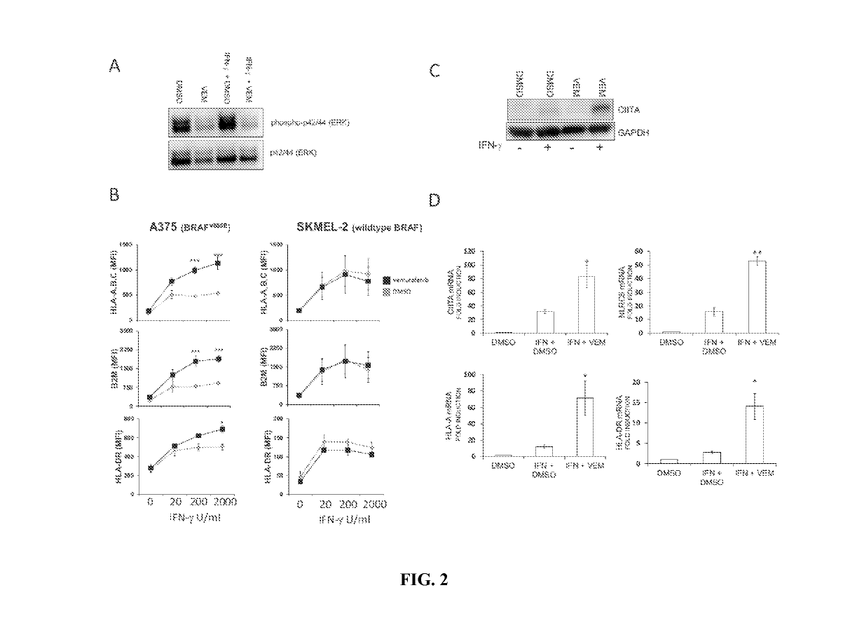

Experiments were performed to determine whether this effect was influenced by the concentration of IFN-γ because the cellular response to IFN-γ can vary with concentration. While PLX4720 is structurally related to vemurafenib it is not used clinically. Therefore, we repeated these experiments using vemurafenib as it has been approved for use in patients whose tumors are BRAFV600E positive. SKMEL-2 cells were included as a control since these cells are wild-type at BRAF codon 600 and thus should be unaffected by selective inhibitors of BRAFV600E such as vemurafenib. As shown in FIG. 2A, like PLX4720, vemurafenib decreased ERK phosphorylation levels in A375 cells alone and when combined with IFN-γ. Consistent with what we observed with PLX4720, vemurafenib enhanced the induction of MHCI and β2-microglobluin (B2M) by IFN-γ in A375 cells (FIG. 2B). In addition, the induction of MHCII molecules was also enhanced by vemurafenib. The effect of vemurafenib was greatest at the higher concentrations of IFN-γ used in this assay (200 U/ml and 2000 U/ml).

In contrast to A375 cells, vemurafenib had no effect on MHC induction in SKMEL-2 cells despite the fact that these cells responded to IFN-γ with increases in cell surface MHCI, MHCII and B2M protein levels (FIG. 2B). Because the induction of both MHCI and MHCII molecules were enhanced, vemurafenib may be increasing IFN-γ-induced proteins that are able to regulate the induction of both MHCI and MHCII molecules such as the MHCII transactivator, CIITA. Vemurafenib increased levels of CIITA protein (FIG. 2C). In addition, vemurafenib increased steady state mRNA levels of CIITA and those of the related transcriptional co-activator NLRC5 in response to IFN-γ (FIG. 2D). Steady state mRNA levels of MHCI (HLA-A) and MHCII (HLA-DR) were also increased by vemurafenib (FIG. 2D) as were levels of gamma-interferon-inducible lysosomal thiol reductase (GILT), an enzyme involved in the processing of some MDAs such as tyrosinase-related protein 1.

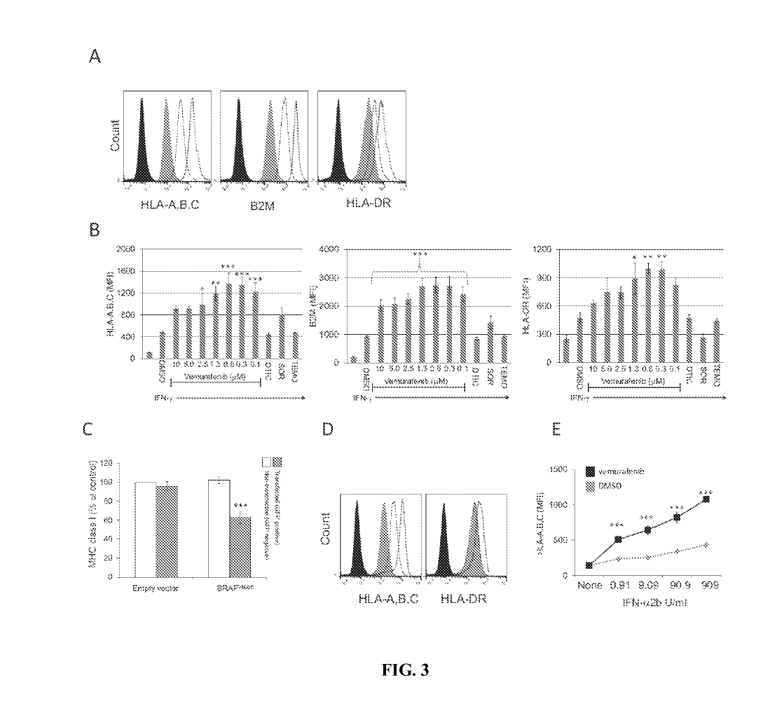

Since the anti-proliferative effect of vemurafenib on A375 cells is optimal at nanomolar concentrations, these experiments were repeated using serial dilutions of vemurafenib from 10 μM to 100 nM. As observed using 10 μM of vemurafenib, lower concentrations of vemurafenib also enhanced the induction of MHCI, B2M and MHCII molecules in response to IFN-γ (FIGS. 3A and 3B). In this model system, the peak enhancement of MHC induction was observed using vemurafenib concentrations of 312 nM and 625 nM though 100 nM was still active in this regard. These concentrations are likely relevant to patients treated with current dosing regimens of vemurafenib (960 mg twice daily) since the mean maximum steady state plasma concentration of vemurafenib have been reported to be 86 μM+/−32 μM. Since vemurafenib is more than 99% protein bound, free concentrations in patients would be expected to be within the concentration ranges used in our in vitro experiments using 10% fetal bovine serum. The kinase inhibitor sorafenib was also included as well as dacarbazine and temozolomide, all of which have been tested in the treatment of metastatic melanoma. Sorafenib enhanced the induction of MHCI molecules yet decreased the induction of the MHCII molecule HLA-DR though none of these differences were statistically significant using a repeated measures analysis of variance (ANOVA). No effect on MHC expression was seen using dacarbazine or temozolomide. Thus in our model system, nanomolar concentrations of vemurafenib can enhance the induction of MHC molecules in A375 cells by IFN-γ.

### Forced Over-Expression of BRAFV600E Represses MHC Class I Levels

Experiments were performed to determine whether the over-expression of BRAFV600E would have the opposite effect of vemurafenib on MHC expression. To this end, A375 cells were transfected with a plasmid encoding BRAFV600E and green fluorescent protein (GFP) on the same transcript or the parental plasmid encoding GFP alone (empty vector). As shown in FIG. 3C, in cells successfully transfected with the plasmid containing BRAFV600E, there was a significant decrease in cell surface MHC class I expression. Thus, forced expression of BRAFV600E can repress basal MHCI levels even in cells already harboring the BRAFV600E mutation.

### Vemurafenib Enhances the Induction of MHC Molecules by IFN-α2b.

Experiments were performed to determine whether vemurafenib can influence MHC induction in response to type I interferons since like IFN-γ (a type II interferon) as these cytokines are potent inducers of MHCI. Experiments were performed to determine whether vemurafenib can enhance MHCI induction in response to IFN-α2b, A375 cells were pre-treated with either vehicle (DMSO) or vemurafenib and then exposed them to increasing concentrations of IFN-α2b. Vemurafenib enhanced the induction of MHCI molecules by IFN-α2b at all the doses utilized which ranged from 0.09 to 909 U/ml (FIGS. 3D and 3E). With regards to MHCII, IFN-α2b had no impact on MHCII expression when used alone yet in the presence of vemurafenib increased MHCII expression in A375 cells (FIG. 3D). Thus, in some cellular contexts, vemurafenib can enhance the induction of MHC molecules in melanoma by IFN-α2b.

### MHC Induction by IFN-γ is Enhanced by Vemurafenib in Cell Lines Homozygous for BRAFV600E but not in Those Heterozygous for BRAFV600E.

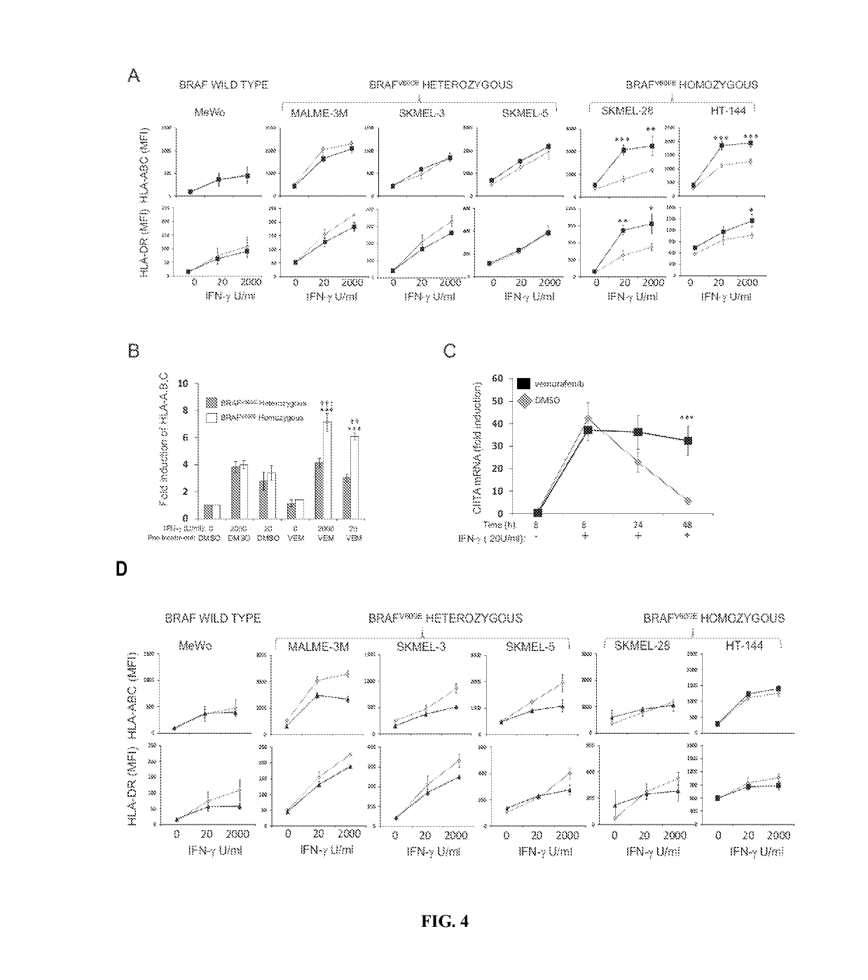

Experiments were performed to determine whether the results obtained with A375 cells could be reproduced in other cellular contexts. These experiments were repeated using additional melanoma cell lines. These included another BRAF wild-type cell line MeWo, as well as cells lines known to harbor the BRAFV600E mutation including MALME-3M, SKMEL-3, SKMEL-5, SKMEL-28, HT-144 and UACC-62. These cell lines were selected since they were all commercially available and because information regarding their mutation status was available using the Wellcome Trust Sanger Institute database which is publically available (http://www.sanger.ac.uk). The BRAF mutation status was confirmed for these cell lines by pyrosequencing BRAF codon 600 and included an additional non-melanoma cell line as a control (A431). For this analysis, the terms wild-type, heterozygous and homozygous only refer to the sequence of BRAF codon 600. Specifically, wild-type cells are those where no mutant sequence is present at codon 600, heterozygous cells are those where both mutant (V600E) and wild-type sequence is detected, and homozygous cells are those where only the mutant (V600E) sequence is detected. Consistent with what was reported in the aforementioned database, MALME-3M, SKMEL-3, and SKMEL-5 cells all harbored both mutant and wild-type sequence at codon 600 of BRAF. In contrast, A375, SKMEL-28 and HT-144 cells possessed only the mutant sequence at codon 600. Experiments were repeated using these cell lines to assess how vemurafenib influenced MHC induction by IFN-γ. Vemurafenib had no impact on the induction of MHCI and MHCII molecules in MeWo cells which are wild-type at BRAF codon 600 (FIG. 4A). Vemurafenib also had no effect on the induction of MHCI and MHCII molecules in MALME-3M, SKMEL3, or SKMEL5 cells all of which are heterozygous for BRAFV600E (FIG. 4A). In contrast, the induction of MHCI molecules in BRAFV600E homozygous SKMEL-28, HT-144 and UACC-62 cells was enhanced by vemurafenib (FIG. 4A). This effect was not due to underlying differences in how heterozygous and homozygous cell lines respond to IFN-γ. Indeed, the fold increase of MHCI levels by IFN-γ was the same (around four fold) for both BRAFV600E homozygous and heterozygous cell lines (FIG. 4B). Rather, vemurafenib enhanced the response to IFN-γ only in cells homozygous for BRAFV600E. Since vemurafenib enhanced the induction of CIITA mRNA in A375 cells which are homozygous for BRAFV600E, experiments were performed to determine whether this was also true in another BRAFV600E homozygous cell line. Vemurafenib increased CIITA steady state mRNA levels following IFN-γ in SKMEL-28 cells (FIG. 4C). The effect of vemurafenib was most pronounced 48 hours after the addition of IFN-γ.

To determine whether this effect was unique to inhibitors targeting the MAPK pathway, how the induction of MHCI and MHCII molecules by IFN-γ is altered in the presence of an inhibitor of the phosphoinositide 3-kinase (PI3K) pathway was examined. BEZ235 a dual PI3K/mammalian target of rapamycin inhibitor was used. While BEZ235 reduced the phosphorylation of AKT (serine 473) in our model system consistent with its ability to inhibit mTOR and PI3K signaling, it did not enhance the induction of MHC molecules by IFN-γ, rather, it attenuated MHC induction in some of the cell lines examined (FIG. 4D).

1. A method of treating cancer comprising:
i) analyzing both chromosomes in a cell from a subject for the a V600E mutation of BRAF; and
ii) determining if both of the chromosomes contain the V600E mutation, then treating the subject comprising the step of administering an effective amount a tyrosine kinase inhibitor in combination with an immunotherapy to the subject.

2. The method of claim 1, wherein the cancer is melanoma.

3. The method of claim 1, wherein the tyrosine kinase inhibitor is vemurafenib, PLX4720, sorafenib, temozolomide, or dabrafenib.

4. The method of claim 1, wherein the immunotherapy is administration of an interferon, administration of an interleukin, administration of an anti-PD1 antibody, administration of an anti-CTLA-4 antibody, a cancer vaccine, or adoptive cell transfer.

5. The method of claim 1, wherein the interferon is human recombinant IFN-α, IFN-α2b or IFN-γ or the interleukin is human recombinant IL-2.

6. The method of claim 1, further comprising the step of recording whether the subject is homozygous for the V600E mutation, heterozygous for the V600E mutation, heterozygous for the wild-type V at 600, or homozygous for the wild-type V at 600.

7. The method of claim 1, wherein the recording is on a computer readable medium.

8. The method of claim 6, further comprising the step of reporting whether the subject is homozygous for the V600E mutation, heterozygous for the V600E mutation, heterozygous for the wild-type V at 600, or homozygous for the wild-type V at 600 to a medical professional, subject, or representative thereof.
